(19) United States
(12) Patent Application Publication
(10) Pub. No.: US 2015/0269752 A1
(43) Pub. Date: Sep. 24, 2015

(54) X-RAY CT APPARATUS AND METHOD FOR IMAGE PROCESSING

(71) Applicants: KABUSHIKI KAISHA TOSHIBA, Minato-Ku (JP); TOSHIBA MEDICAL SYSTEMS CORPORATION, Otawara-Shi (JP)

(72) Inventor: Masahiko YAMAZAKI, Utsunomiya (JP)

(73) Assignees: KABUSHIKI KAISHA TOSHIBA, Minato-Ku (JP); TOSHIBA MEDICAL SYSTEMS CORPORATION, Otawara-Shi (JP)

(21) Appl. No.: 14/636,439

(22) Filed: Mar. 3, 2015

(30) Foreign Application Priority Data
Mar. 18, 2014 (JP) 2014-055420

(51) Int. Cl. G06T 11/00; A61B 6/00; A61B 6/03

(52) U.S. Cl. CPC G06T 11/003; A61B 6/032; A61B 6/5205; A61B 6/583; G06T 2207/10004; G06T 2207/10081

(57) ABSTRACT

The X-ray CT apparatus includes an X-ray tube, an X-ray detector, a rotation unit, a data generation unit, an image generation unit and a setting unit. The data generation unit generates data used in reconstruction processing of a CT image on a basis of an output of the X-ray detector. The image generation unit performs reconstruction processing of the CT image, the reconstruction processing using the data and a parameter value relating to at least one of an FDD and an FCD. The setting unit sets the parameter value used in the reconstruction processing using the data generated when the X-ray is emitted to the object, the parameter value being set based on CT values of a plurality of CT images obtained by a plurality of parameter values used in the reconstruction processing using the data generated when the X-ray is emitted to a phantom.

### CROSS-REFERENCE TO RELATED APPLICATION

This application is based upon and claims the benefit of priority from Japanese Patent Application No. 2014-55420, filed on Mar. 18, 2014, the entire contents of which are incorporated herein by reference.

### FIELD

An embodiment as one aspect of the present invention relates to an X-ray computed tomography (CT) apparatus and a method for image processing.

### BACKGROUND

The X-ray CT apparatus provides information on an object in the form of images based on intensity of an X-ray which passed the object. In a large number of medical practices including diagnosis and treatment of diseases and planning of surgical operations, the X-ray CT apparatus plays an important role.

One form of the X-ray CT apparatus is a rotate-rotate geometry (R/R geometry, third generation scanner). In the R/R geometry, an X-ray tube and an X-ray detector are arranged to face each other with an object spatially interposed therebetween. The X-ray tube and X-ray detector integrally rotate around the object and collect data. More specifically, when the X-ray tube and the X-ray detector integrally rotate around the object, projection data in each view (angle of rotation) is collected at every fixed angle (sampling point).

The X-ray CT apparatus of R/R geometry has an advantage over apparatuses of other geometries in terms of a scattered beam removal capacity, economical efficiency and the like. Accordingly, most of the X-ray CT apparatuses currently in use employ the R/R geometry. There is disclosed an X-ray CT apparatus of R/R geometry which can obtain high-resolution reconstruction images by shifting a position of an X-ray source with respect to a rotational frame.

In the X-ray CT apparatus of R/R geometry, images are reconstructed under assumption that actual geometrical values representing an X-ray focal point of the X-ray tube and positions of detection elements and the like are equal to design values. However, actual positions of the X-ray focal point of the X-ray tube, the detection elements and the like, are deviated from the design values for use in reconstruction, which causes a problem of deteriorated resolution of CT images.

### BRIEF DESCRIPTION OF THE DRAWINGS

In accompanying drawings,

FIG. 1 is a configuration example illustrating an X-ray CT apparatus according to a present embodiment;

FIG. 2 is a block diagram illustrating functions of the X-ray CT apparatus according to the present embodiment;

FIG. 3 is one example of a bead phantom;

FIG. 4 is one example of rotation conversion;

FIG. 5 is a flow chart illustrating an operation for calculating a back projection parameter optimum value;

FIG. 6 is a relationship between FDD values and corresponding bead CT values;

FIG. 7 is an explanatory view illustrating generation of CT images for each detector module;

FIG. 8 is one example of a line phantom;

FIG. 9A is one example of a coin phantom;

FIG. 9B is a graph view illustrating a coin CT value in each of the slices; and

FIG. 10 is one example of a cube phantom.

### DETAILED DESCRIPTION

The X-ray CT apparatus and the method for image processing according to the present embodiment(s) are described with reference to accompanying drawings.

To solve the above-described problems, the present embodiment provides the X-ray CT apparatus, including: an X-ray tube configured to emit an X-ray; an X-ray detector configured to detect the X-ray; a rotation unit configured to rotate at least the X-ray tube around a rotation center; a data generation unit configured to generate data used in reconstruction processing of a CT image on a basis of an output of the X-ray detector; an image generation unit configured to perform reconstruction processing of the CT image, the reconstruction processing using the data and a parameter value relating to at least one of a distance (FDD) between a focal position of the X-ray tube and a position of the X-ray detector, and a distance (FCD) between the focal position of the X-ray tube and a position of the rotation center; and a setting unit configured to set the parameter value used in the reconstruction processing using the data generated when the X-ray is emitted to the object, the parameter value being set based on CT values of a plurality of CT images obtained by a plurality of parameter values used in the reconstruction processing using the data generated when the X-ray is emitted to a phantom.

To solve the above-described problems, the present embodiment provides the X-ray CT apparatus, including: an X-ray tube configured to emit an X-ray; an X-ray detector configured to detect the X-ray emitted by the X-ray tube; a data generation unit configured to generate data used in reconstruction processing of a CT image on a basis of an output of the X-ray detector; an image generation unit configured to perform reconstruction processing of the CT image, the reconstruction processing using the data and a parameter value; and a setting unit configured to set the parameter value used in the reconstruction processing using the data generated when the X-ray is emitted to the object, the parameter value being set based on CT values of a plurality of CT images obtained by a plurality of parameter values used in the reconstruction processing using the data generated when the X-ray is emitted to a phantom.

To solve the above-described problems, the present embodiment provides the method for image processing, including: obtaining data used in reconstruction processing of a CT image from a storage, the data being acquired by a CT scan; performing reconstruction processing of the CT image, the reconstruction processing using the data and a parameter value relating to at least one of a distance (FDD) between a focal position of an X-ray tube configured to emit an X-ray and a position of an X-ray detector configured to detect the X-ray, and a distance (FCD) between the focal position of the X-ray tube and a position of the rotation center; and setting a parameter value used in the reconstruction processing using the data generated when the X-ray is emitted to the object, the parameter value being set based on CT values of a plurality of CT images obtained by a plurality of parameter values used in the reconstruction processing using the data generated when the X-ray is emitted to a phantom.

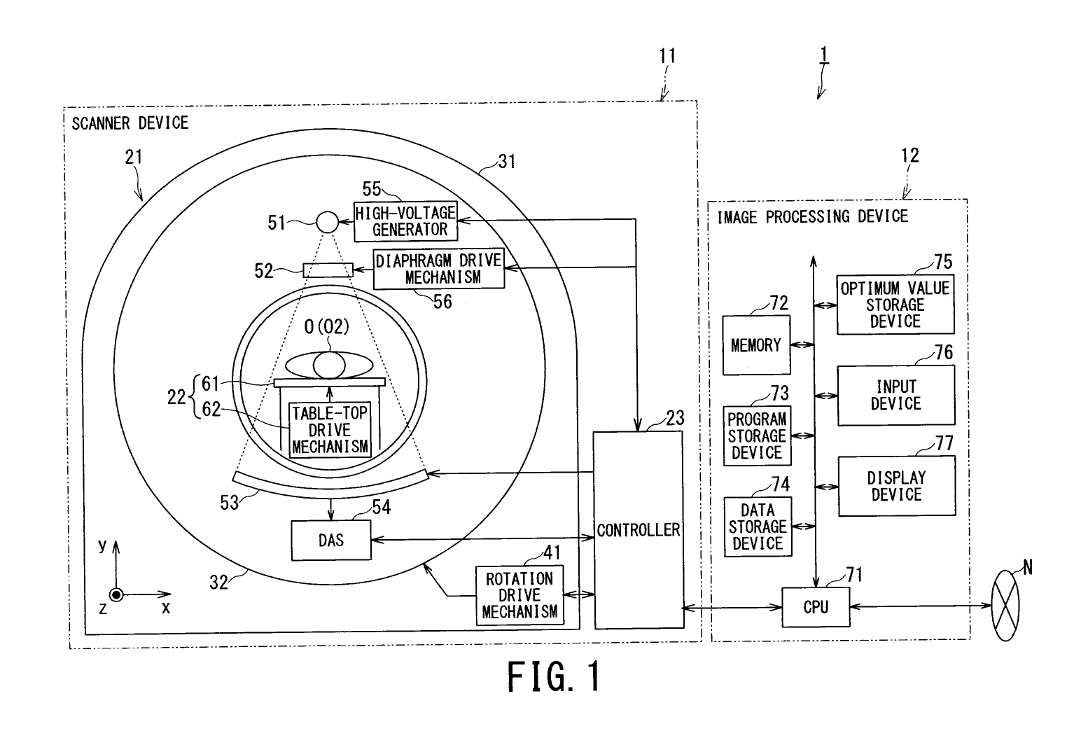

FIG. 1 is a configuration example illustrating an X-ray CT apparatus according to the present embodiment.

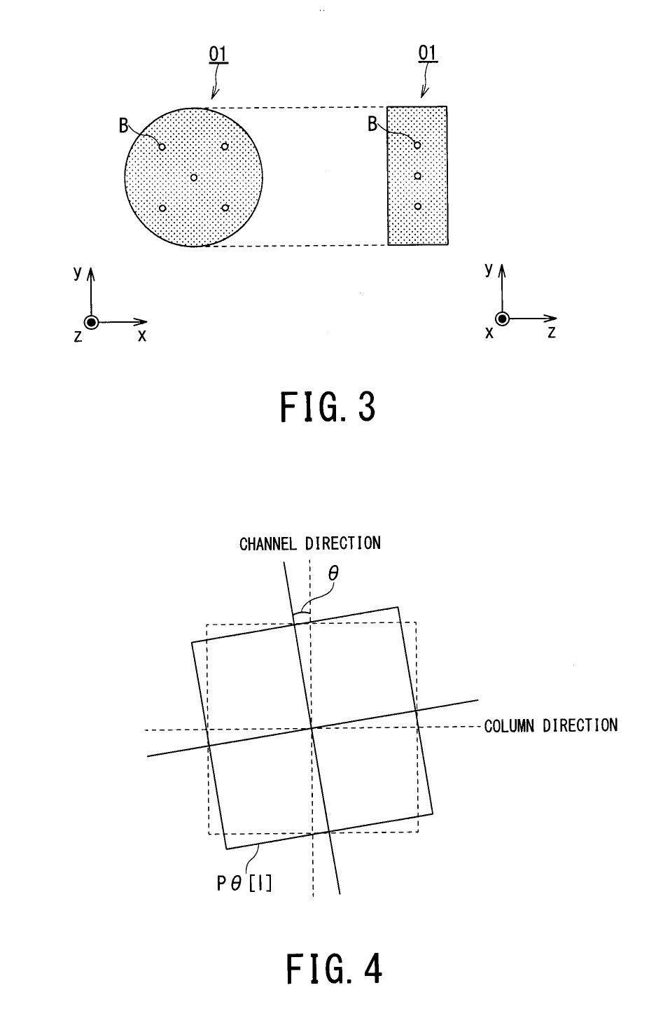

FIG. 1 illustrates an X-ray CT apparatus 1 of R/R geometry according to the present embodiment. The X-ray CT apparatus 1 is mainly constituted of a scanner device 11 and an image processing device (console) 12. The scanner device 11 in the X-ray CT apparatus 1 is typically placed in a laboratory and is configured to generate X-ray transmission data on an object O (a phantom O1 (illustrated in FIG. 3) or a patient O2). The image processing device 12 is typically placed in a control room adjacent to the laboratory and is configured to generate projection data based on the transmission data and to generate and display reconstructed images.

The scanner device 11 in the X-ray CT apparatus 1 includes a gantry device 21, a bed device 22, and a controller (processing circuit) 23.

The gantry device 21 in the scanner device 11 includes a fixed frame 31 fixed to a base portion (not illustrated), and a rotational frame 32.

The fixed frame 31 includes a rotation drive mechanism 41. The rotation drive mechanism 41 has a mechanism to rotate the rotational frame 32 with respect to the fixed frame 31 around an opening portion including a rotation center while a position relation of the rotational frame 32 is maintained under the control of the controller 23.

The rotational frame 32 integrally holds an X-ray source (X-ray tube) 51, a diaphragm 52, an X-ray detector 53, a data acquisition system (DAS) 54, a high-voltage generator 55, and a diaphragm drive mechanism 56. The rotational frame 32 is configured to enable the X-ray tube 51, the diaphragm 52, the X-ray detector 53, the DAS 54, the high-voltage generator 55, and the diaphragm drive mechanism 56 to integrally rotate around the object O while making the X-ray tube 51 and the X-ray detector 53 face each other. A direction parallel to a central axis of rotation of the rotational frame 32 is defined as a z-axis direction, and a plane perpendicular to the z-axis direction is defined to extend in an x-axis direction and a y-axis direction.

The X-ray tube 51 generates an X-ray (continuous spectrum X-ray) because an electron beam collides against a metal target in accordance with a tube voltage supplied from the high-voltage generator 55, and emits the generated X-ray to the X-ray detector 53. The X-ray emitted from the X-ray tube 51 forms a fan beam X-ray and/or a cone beam X-ray. Under the control of the controller 23 via the high-voltage generator 55, the X-ray tube 51 receives a supply of power necessary for X-ray emission.

The diaphragm 52 adjusts an irradiation area (irradiation field) of the X-ray emitted from the X-ray tube 51 under the control of the diaphragm drive mechanism 56. More specifically, the X-ray irradiation area in a slice direction can be changed by adjusting an aperture of the diaphragm 52 by the diaphragm drive mechanism 56.

The X-ray detector 53 is a one-dimensional array-type detector having a plurality of detection elements in a channel direction and a single detection element in a column (slice) direction. Or the X-ray detector 53 is a two-dimensional array-type detector (also referred to as a multi-slice detector) having detection elements in a matrix form, i.e., having a plurality of detection elements in the channel direction and a plurality of detection elements in the slice direction. When the X-ray detector 53 is a multi-slice detector, a three-dimensional photographing area having a width in a column direction can be photographed in one rotation scan (volume scan). The X-ray detector 53 detects an X-ray emitted from the X-ray tube 51 under the control of the controller 23.

The DAS 54 amplifies a signal representative of transmission data (X ray detection data) detected by each detection element of the X-ray detector 53, and converts the signal into a digital signal. Output data of the DAS 54 is supplied to the image processing device 12 through the controller 23 of the scanner device 11. The DAS 54 will be described later in detail.

The high-voltage generator 55 supplies electric power necessary to execute a scan to the X-ray tube 51 under the control of the controller 23.

The diaphragm drive mechanism 56 has a mechanism of adjusting an X-ray irradiation area in the slice direction in the diaphragm 52 under the control of the controller 23.

The bed device 22 of the scanner device 11 includes a table-top 61 and a table-top drive mechanism 62. The object O can be laid on the table-top 61.

The table-top drive mechanism 62 has a mechanism of moving the table-top 61 up and down along a y-axis direction and also advancing/retracting the table-top 61 along a z-axis direction under the control of the controller 23. The table-top drive mechanism 62 inserts the object O laid on the table-top 61 into the opening portion including the rotation center of the rotational frame 32, and retracts the object O laid on the table-top 61 from the opening portion.

The controller 23 of the scanner device 11 includes a central processing unit (CPU) as an unillustrated processing circuit and a memory. In response to an instruction from the image processing device 12, the controller 23 controls the rotation drive mechanism 41, the X-ray detector 53, the DAS 54, the high-voltage generator 55 and the diaphragm drive mechanism 56 of the gantry device 21, and/or the table-top drive mechanism 62 and the like of the bed device 22 to execute a scan.

The image processing device 12 of the X-ray CT apparatus 1, which has a computer-based configuration, can mutually communicate with a network (local area network) N.

The image processing device 12 is not limited to being included in the X-ray CT apparatus 1. The image processing device 12 may be an off-line device independent of the X-ray CT apparatus 1. In that case, the image processing device 12 can acquire pre-reconstruction data, such as projection data on the patient O2 collected by the X-ray CT apparatus 1 via a portable storage medium (media). Or the image processing device 12 may be an online device independent of the X-ray CT apparatus 1. In that case, the image processing device 12 is provided on a medical image system connected to various devices such as unillustrated image management devices (image servers) and/or unillustrated diagnostic reading terminals via the network. The image processing device 12 can acquire the pre-reconstruction data, such as projection data on the patient O2 collected by the X-ray CT apparatus 1, from the image management devices and the like.

Furthermore, when the image processing device 12 is an online device independent of the X-ray CT apparatus 1, later-described functions in the image processing device 12 may be distributed to devices each constituting the medical image system, so that an entire medical image system implements the present invention.

The image processing device 12 is mainly constituted of basic hardware components including a CPU 71 as a control circuit, a memory 72, a program storage device 73, a data storage device 74, an optimum value storage device 75, an input device 76, and a display device 77. The CPU 71 is mutually connected to respective hardware components that constitute the image processing device 12 via a bus used as a common signal transmission line.

The CPU 71 is a hard disk drive (HDD) having a packaged integrated circuit (LSI) structure in which an electronic circuit made of a semiconductor has a plurality of terminals.

When an operator such as a doctor operates the input device 76, and a command is input, the CPU 71 executes a program stored in the memory 72. Or the CPU 71 loads a program which is stored in the program storage device 73 or a program which is transferred from the network N and installed in the program storage device 73, to the memory 72 for execution.

The memory 72 is a storage device including a read only memory (ROM) and a random access memory (RAM). The memory 72 is used to store an initial program loading (IPL), a basic input/output system (BIOS) and data, and is also used as a work memory of the CPU 71 and/or a temporal storage of data.

The program storage device 73 is a storage device configured to unremovably incorporate a metal disc having magnetic substances applied or vapor-deposited thereto. The program storage device 73 is a storage device that stores programs (including application programs and an operating system (OS)) installed in the image processing device 12 and data. The program storage device 73 also enables the OS to provide a graphical user interface (GUI) which allows heavy use of graphics to display information on the display device 77 for an operator such as a surgical operator and allows basic operations to be performed with the input device 76.

The data storage device 74 is constituted of a HDD configured to unremovably incorporate a metal disc having magnetic substances applied or vapor-deposited thereto or storage media drive which can receive and remove a portable storage medium (medium). The data storage device 74 stores pre-reconstruction data such as projection data on the patient O2, and/or CT image data on the patient O2.

The optimum value storage device 75 is constituted of a HDD configured to unremovably incorporate a metal disc having magnetic substances applied or vapor-deposited thereto and/or a storage media drive which can receive and remove a portable storage medium (medium). The optimum value storage device 75 stores a later-described back projection parameter optimum value for each of back projection parameter items. The back projection parameter optimum value is calculated based on CT values of CT images on a phantom obtained by scanning the phantom.

The input device 76 is a pointing device which can be operated by an operator. An input signal in conformity to an operation is sent to the CPU 71.

The display device 77 includes an image synthesis circuit, a video random access memory (VRAM), and a display not illustrated. The image synthesis circuit generates synthesis data by synthesizing image data and text data of various display parameters or the like. The VRAM expands the synthesis data on the display. The display is constituted of a liquid crystal display, a cathode ray tube (CRT), or the like, to sequentially display images.

The image processing device 12 applies logarithmic conversion processing and correction processing (preprocessing), such as sensitivity correction, to raw data input from the DAS 54 of the scanner device 11 to generate projection data, and stores the generated data in the data storage device 74. The image processing device 12 also performs processing to eliminate scattered beams in pretreated projection data. The image processing device 12 performs scattered beam elimination based on values of the projection data in an X-ray exposure range. Scattered beams are estimated based on the magnitude of values representative of projection data that is a target of scattered beam correction or values representative of projection data adjacent to the target projection data. The estimated scattered beams are then subtracted from the target projection data, by which the scattered beam correction is performed. The image processing device 12 generates CT image data on the object O based on the corrected projection data. The image processing device 12 then stores the generated data in the data storage device 74 and/or display the data on the display device 77.

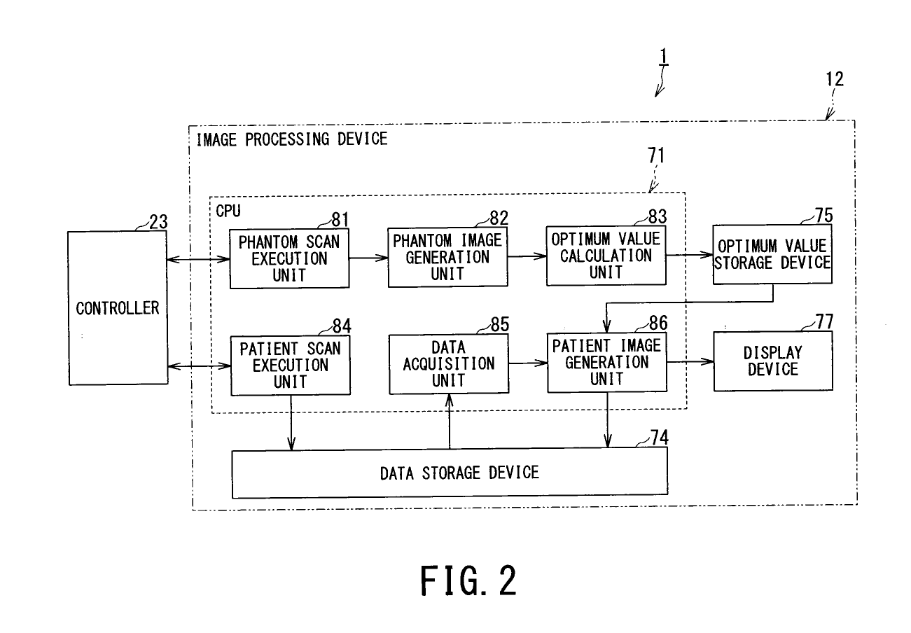

FIG. 2 is a block diagram illustrating functions of the X-ray CT apparatus 1 according to the present embodiment.

When the CPU 71 in the image processing device 12 illustrated in FIG. 1 executes a program, the X-ray CT apparatus 1 functions as a phantom scan execution unit 81, a phantom image generation unit 82, an optimum value calculation unit 83, a patient scan execution unit 84, a data acquisition unit 85, and a patient image generation unit 86 as illustrated in FIG. 2. All or part of the units 81 to 86 may be included as a hardware component(s) such as a circuit(s) in the image processing device 12. All or part of the units 81 to 86 may be included not only in the image processing device 12 but also in the high-voltage generator 55 and/or the controller 23.

The phantom scan execution unit 81 has a function of controlling the scanner device 11 via the controller 23 to cause the scanner device 11 to execute a scan of the phantom O1 and generating pre-reconstruction data such as projection data. The phantom O1 contains at least two kinds of substances (a reference substance and a surrounding substance surrounding the reference substance) which are different in an absorption coefficient (linear absorption coefficient). Here, the absorption coefficient represents a level of X-ray absorption. The absorption coefficient is measured with a crystal spectroscope or a grating spectrograph while energy of an X-ray emitted to the substance is varied. The phantom O1 may also contain at least two kinds of substances which are identical in the absorption coefficient and different in density (mass absorption coefficient).

FIG. 3 is one example of a bead phantom O1.

FIG. 3 includes a cross sectional view on a left-hand side illustrating the bead phantom O1 along x-y direction, and a cross sectional view on a right-hand side illustrating the bead phantom O1 along y-z direction. As illustrated in FIG. 3, the bead phantom O1 contains a plurality of spherical beads B including a central bead as a plurality of reference substances. When the phantom is scanned, the central bead among the plurality of beads B of the bead phantom O1 is preferably arranged as a rotation center.

The beads B and their surrounding substance in the bead phantom O1 are so set that the beads B are larger (or smaller) in the absorption coefficient than the surrounding substance. Hereinafter, unless otherwise specified, the case where the beads B are larger in the absorption coefficient than the surrounding substance will be described as an example.

With reference again to FIG. 2, the phantom image generation unit 82 has a function of performing convolution calculation and/or interpolating calculation based on the pre-reconstruction data generated by the phantom scan execution unit 81. The phantom image generation unit 82 also has a function of generating, each of a plurality of CT images on the phantom O1 by performing back projection calculation (back projection) using a plurality of back projection parameter values with respect to back projection parameter items, and the pre-reconstruction data subjected to convolution calculation and interpolating calculation. The back projection parameter items refer to various parameter items for use in back projection calculation.

Here, the back projection parameter items include at least one of a distance (FDD: focus detector distance) between a focal position of the X-ray tube 51 and the X-ray detector 53 (illustrated in FIG. 1), and a distance (FCD: focus center distance) between the focal position of the X-ray tube 51 and the center of rotation.

As the back projection parameter item, at least one item may be added from a group including a channel direction position of each detection element constituting the X-ray detector 53, an X-ray incidence direction position of each detection element constituting the X-ray detector 53, a column direction position of each detection element constituting the X-ray detector 53, an in-plane rotation direction position of each detection element constituting the X-ray detector 53, and a focal point size of the X-ray tube 51. The phantom image generation unit 82 generates a plurality of CT images for each back projection parameter item.

When each back projection parameter item takes a plurality of different back projection parameter values, a plurality of CT images different in CT value can be obtained by the phantom image generation unit 82. This will be described with Expressions (1) to (3) which are discrete Expressions for back projection calculation.

$$image ( x , y , k ) = {\sum }_{n = {n}_{1} ( z )}^{{n}_{g} ( z )} \frac{P ( n , ch , seg )}{{L}^{2} ( β , x , y )} \qquad ( 1 )$$

In Expression (1), a value “image (x, y, k)” represents a CT value of each pixel (x, y) in a CT image of a slice at k position. A value “P (n, ch, seg)” represents projection data P in a channel “ch” and a column “seg” subjected to convolution calculation and interpolating calculation. A value “L (β, x, y)” represents a distance between the X-ray focal point at a rotation angle β and a voxel position (x, y, z_{fs}). The position (x, y, z_{fs}) is a z-axis coordinate of the X ray focal point.

The positions in the channel direction “ch” and in the column direction “seg” in Expression (1) are expressed by Expressions (2) and (3). In Expression (2), a value “ch0” represents a center position of the X-ray detector 53 in the channel direction. A value “seg0” in Expression (3) represents a center position of the X-ray detector 53 in the column direction.

$$\begin{matrix}ch = \frac{1}{Δγ} {sin}^{- 1} \{ \frac{x cos β + y sin β}{L ( β , x , y )} \} + ch 0 & ( 2 ) \\ seg = ζ ( z ) \frac{FCD}{wL ( β , x , y )} + seg 0 & ( 3 )\end{matrix}$$

First, the FDD as a back projection parameter item is described. The FDD value as a back projection parameter value relates to Expression (2). A value “Δγ” in Expression (2) is an angular distance (pitch) between two detection elements adjacent in the channel direction in X-ray detector 53 (illustrated in FIG. 1). Accordingly, the angular distance Δγ is expressed by Expression (4) by using a designed value Δch of an entire angular distance of the X-ray detector 53. Therefore, when a plurality of different FDD values are taken, a plurality of CT images different in CT value can be obtained by Expression (4):

$$Δγ = \frac{Δ ch}{FDD \times 2 z} [ rad ] \qquad ( 4 )$$

Next, a description is now given of the FCD as a back projection parameter item. The FCD value which is a back projection parameter value is expressed by “FCD” in Expression (3). Therefore, when a plurality of different FCD values are taken, a plurality of different CT images different in CT value may be obtained by Expression (1).

Now, a description is given of the channel direction position of each detection element constituting the X-ray detector 53 as a back projection parameter item. A channel direction position Pch[i] of a detection element in the i-th channel as a back projection parameter value relates to Expression (2). While the angular distance Δγ is constant in Expression (2), Expression (2) may be modified to have the angular distance Δγ varied in each of the plurality of detection elements. More specifically, “Δγ” in Expression (2) can be replaced with “Δγ[i]” which is varied by i. Therefore, when the plurality of detection elements take different channel direction positions Pch[i], a plurality of CT images different in CT value can be obtained by Expression (1). Instead of varying the channel direction position with respect to each channel, the channel direction position may be varied with respect to each detection element of the X-ray detector 53 or each detector module of detector modules obtained by dividing the X-ray detector 53 in the channel direction.

Now, a description is given of the X-ray incidence direction position of each detection element constituting the X-ray detector 53 as a back projection parameter item. An X-ray incidence direction position Pw[j] of a j-th detection element as a back projection parameter value relates to Expression (3). Since a value “w” in Expression (3) is a value of a slice thickness of the X-ray detector 53 projected on the central axis of rotation, Expression (3) can be modified into Expression (5) by using a designed value Pw of the X-ray incidence direction position. Therefore, when the plurality of detection elements take different X-ray incident positions Pw[j], a plurality of CT images different in CT value can be obtained by Expression (1).

$$w [ j ] = \frac{Pw}{Pw [ j ]} \times w \qquad ( 5 )$$

Now, a description is given of the column direction position of each detection element constituting the X-ray detector 53 as a back projection parameter item. A column direction position Ps[k] of a detection element in the k-th column as a back projection parameter value relates to Expression (6) expanded from Expression (3). Expression (6) is a modification of Expression (3) with use of a designed value P sin the column direction. Therefore, when the plurality of detection elements take different column direction positions Ps[k], a plurality of CT images different in CT value can be obtained by Expression (1). Instead of varying the column direction position in unit of columns, the position of the X-ray detector 53 may be varied in unit of detection elements.

$$seg = ζ ( z ) \frac{FCD}{wL ( β , x , y )} + seg 0 + ( Ps [ k ] - Ps ) \qquad ( 6 )$$

Now, a description is given of the in-plane rotation direction position of each detection element constituting the X-ray detector 53 as a back projection parameter item. An in-plane rotation direction position Pθ[l] of an l-th detection element as a back projection parameter value corresponds to rotation-converted positions Pch[i] and Ps[k] as in Expressions (7) and (8). One example of rotation conversion of each detection element to a rotation direction position Pθ[l] is illustrated in FIG. 4. Therefore, when the plurality of detection elements take different rotation direction positions Pθ[l], a plurality of CT images different in CT value can be obtained by Expression (1).

Pch[i]′=Pch[i] cos Pθ[l]−Ps[k] sin Pθ[l] (7)

Ps[k]′=Pch[i] sin Pθ[l]+Ps[k] cos Pθ[l](8)

Finally, the focal point size of the X-ray tube 51 as a back projection parameter item is described. A focal point size Fch of the X-ray tube 51 in the channel direction and a focal point size Fs in the column direction as back projection parameter value relate to Expression (9) expanded from Expression (1). As expressed by Expression (9), CT images are obtained by integrating the focal point size. Therefore, when a plurality of different focal point sizes Fch and Fs are taken, a plurality of CT images different in CT value can be obtained by Expression (1).

$${\int }_{\frac{- Fs}{2}}^{\frac{+ Fs}{2}} {\int }_{\frac{- Fch}{2}}^{\frac{+ Fch}{2}} \frac{image ( x , y , z ) d ( Fch ) \cdot d ( Fs )}{( Fch \times Fs )} \qquad ( 9 )$$

The optimum value calculation unit 83 has a function of calculating a bead CT value based on CT images corresponding to each of the back projection parameter values with respect to a back projection parameter item generated by the phantom image generation unit 82. The optimum value calculation unit 83 also has a function of calculating a back projection parameter value corresponding to a substantially maximum (substantially peak) bead CT value based on the bead CT values corresponding to the respective back projection parameter values, and setting the calculated value as an optimum value of the back projection parameter item (back projection parameter optimum value).

In that case, the bead CT value corresponding to each of the back projection parameter values is one of a maximum value among the plurality of CT values concerning all the bead portions in each of the CT images, a total value or an average value of the plurality of CT values concerning all the bead portions in each of the CT images, a maximum value of the plurality of CT values concerning the bead portions in a set area in each of the CT images, and a total value or an average value of the plurality of CT values concerning all the bead portions in the set area.

When the beads B and their surrounding substance in the bead phantom O1 are so set that the beads B are smaller in the absorption coefficient than the surrounding substance, the optimum value calculation unit 83 calculates a back projection parameter value corresponding to a substantially minimum (substantially peak) CT value, based on the bead CT values corresponding to each of the back projection parameter values.

In that case, the bead CT value corresponding to each of the back projection parameter values is one of a minimum value among the plurality of CT values concerning all the bead portions in each of the CT images, a total value or an average value of the plurality of CT values concerning all the bead portions in each of the CT images, a minimum value of the plurality of CT values concerning the bead portions in the set area in each of the CT images, and a total value or an average value of the plurality of CT values concerning all the bead portions in the set area.

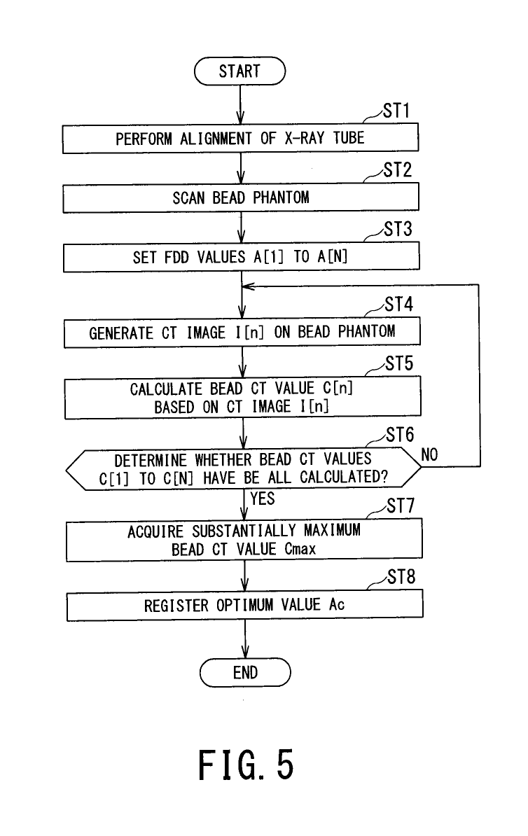

Here, operation of the units 81 to 83 in the case where the back projection parameter item is the FDD will be described with reference to FIG. 5.

FIG. 5 is a flow chart illustrating an operation for calculating the back projection parameter optimum value.

First, the X-ray CT apparatus 1 performs alignment of the X-ray tube 51 (step ST1) and then scans the bead phantom O1 (step ST2).

The X-ray CT apparatus 1 sets FDD values A[1] to A[N] as back projection parameter values so that the pre-reconstruction data on the bead phantom O1 obtained by performing a scan includes FDD design values A as a design value of the back projection parameter item (back projection parameter design value) (step ST3).

The X-ray CT apparatus 1 applies convolution calculation and interpolating calculation to the pre-reconstruction data obtained by performing a scan in step ST2. By performing the back projection calculation using the FDD values A [n (n=1, 2, . . . , N)] as the FDD in Expression (4), the X-ray CT apparatus 1 generates an image I[n] on the bead phantom O1 (step ST4). The X-ray CT apparatus 1 calculates a bead CT value C[n] based on the CT image I[n] on the bead phantom O1 generated in step ST4 (step ST5).

The X-ray CT apparatus 1 determines whether or not bead CT values C[1] to C[N] are all calculated in step ST 5 for each of all the FDD values A[1] to A[N] set in step ST3 (step ST6). When NO is determined in step ST6, that is, when it is determined that the bead CT value is not calculated for at least one of the FDD values A[1] to A[N], the X-ray CT apparatus 1 returns to operation of step ST4.

When YES is determined in step ST6, that is, when it is determined that the bead CT values C[1] to C[N] are calculated for each of all the FDD values A[1] to A[N], the X-ray CT apparatus 1 acquires a substantially maximum bead CT value Cmax from the bead CT values C[1] to C[N] (step ST7).

The X-ray CT apparatus 1 calculates a FDD value corresponding to the substantially maximum bead CT value Cmax acquired in step ST7, and registers it as an FDD optimum value Ac (step ST8).

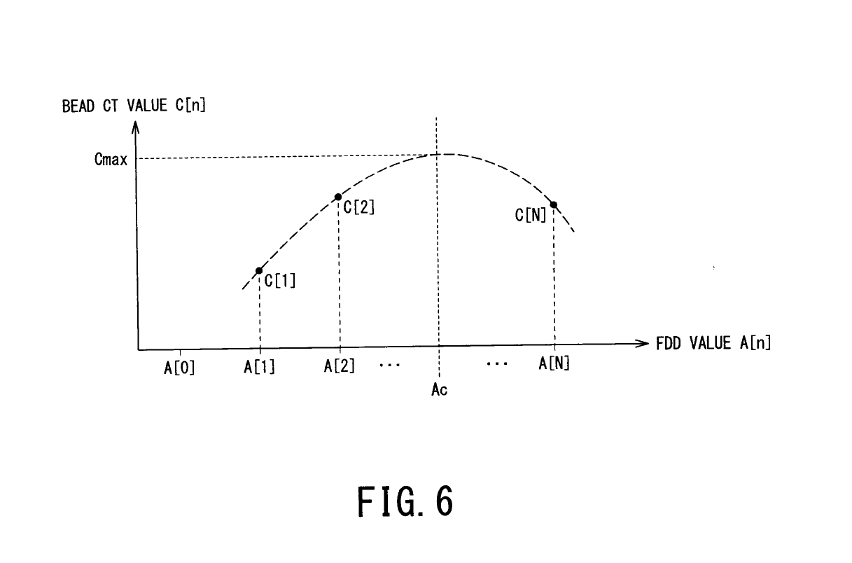

FIG. 6 is a relationship between FDD values A[1] to A[N] and corresponding bead CT values C[1] to C[N].

FIG. 6 is a graph view of an approximated curve obtained by plotting the bead CT values C[1] to C[N] for each of the FDD values A[1] to A[N]. The substantially maximum bead CT value Cmax is obtained from the graph view of FIG. 6.

With reference again to FIG. 5, the operation of steps ST4 to ST6 is repeated, so that an identical value (a design value or an optimum value) is applied to the back projection parameter items other than the FDD, while a plurality of CT images corresponding to a plurality of FDD values are generated only with respect to the FDD.

While an example of calculating the FDD optimum value as a back projection parameter optimum value is described in FIG. 5, other back projection parameter optimum values may similarly be calculated.

With reference again to FIG. 2, the patient scan execution unit 84 has a function of controlling the scanner device 11 via the controller 23 to cause the scanner device 11 to execute a scan of the patient O2 and generating pre-reconstruction data such as projection data. The patient scan execution unit 84 also has a function of storing the pre-reconstruction data in the data storage device 74.

The data acquisition unit 85 has a function of acquiring (reading) the pre-reconstruction data concerning the patient O2 stored in the data storage device 74 by the patient scan execution unit 84.

The patient image generation unit 86 has a function of performing convolution calculation and interpolating calculation, based on the pre-reconstruction data acquired by the data acquisition unit 85. The patient image generation unit 86 also has a function of performing back projection calculation using the back projection parameter optimum values registered in the optimum value storage device 75 and the projection data subjected to convolution calculation and interpolating calculation, and thereby generating CT images on the patient O2. The patient image generation unit 86 also stores the CT images on the patient O2 in the data storage device 74, and/or displays the images on the display device 77.

The patient image generation unit 86 can perform, when the optimum values of all the back projection parameter items are registered in the optimum value storage device 75, back projection calculation using the optimum values of all the back projection parameter items. When the optimum values of only part of the back projection parameter items are registered in the optimum value storage device 75, the patient image generation unit 86 performs back projection calculation using the optimum values of the part of the back projection parameter items and design values of other back projection parameter items.

In the case where a back projection parameter optimum value is changed via the input device 76 (illustrated in FIG. 1) after the patient image generation unit 86 displays the CT image on the patient O2 on the display device 77, the patient image generation unit 86 can generate a CT image using the changed back projection parameter value and display the image on the display device 77.

In the reconstruction processing based on the data on the patient O2, the X-ray CT apparatus 1 according to the present embodiment generates a CT image on the patient O2 by using the back projection parameter optimum value and the data on the bead phantom O1. Therefore, the X-ray CT apparatus 1 can enhance a plane resolution of the CT image on the patient O2.

In the reconstruction processing based on the data on the patient O2, the X-ray CT apparatus 1 generates a CT image by using the back projection parameter optimum value based on the CT value of an image portion (bead portion) in the CT image on the bead phantom O1. Therefore, according to X-ray CT apparatus 1, the back projection parameter optimum value can be calculated by using only the CT value of the reference substance (bead). Accordingly, as compared with the case of calculating the back projection parameter optimum value by using the CT value of the entire CT image, the plane resolution of the CT image on the patient O2 can be enhanced with high precision.

### (First Modification)

The optimum value calculation unit 83 illustrated in FIG. 2 may calculate the back projection parameter optimum value which changes depending on the angle of rotation caused by the rotational frame 32 (illustrated to FIG. 1), based on pre-reconstruction data. If transition of a change in the back projection parameter optimum value in one rotation is determined, the transition value can repeatedly be used for every rotation of the rotational frame 32 (one rotation in one period). Or if transition of a change in the back projection parameter optimum value in half a rotation is determined, the transition value can repeatedly be used for every half rotation of the rotational frame 32 (one rotation in two periods).

In this case, a change portion (correction value) ΔA of the FDD optimum value Ac can be calculated as a back projection parameter optimum value at every rotation angle β. As expressed by Expression (10), the correction value ΔA is composed of four components: an amplitude (Amp.1) and a phase φ1 in the case of one rotation in one period, and an amplitude (Amp.2) and a phase φ2 in the case of one rotation in two periods.

ΔA=[Amp.1] sin(β+φ1)+[Amp.2] cos(2β+φ2) (10)

A value obtained by arbitrarily varying the four components in Expression (10) is defined as a correction value ΔA of the FDD optimum value Ac at every rotation angle β. The rotation angle may be applied to a tilt angle of the gantry device 21 (illustrated in FIG. 1).

### (Second Modification)

When the optimum value calculation unit 83 illustrated in FIG. 2 calculates a plurality of bead CT values each based on a plurality of CT images, the bead CT value in each CT image may be selected for each detector module.

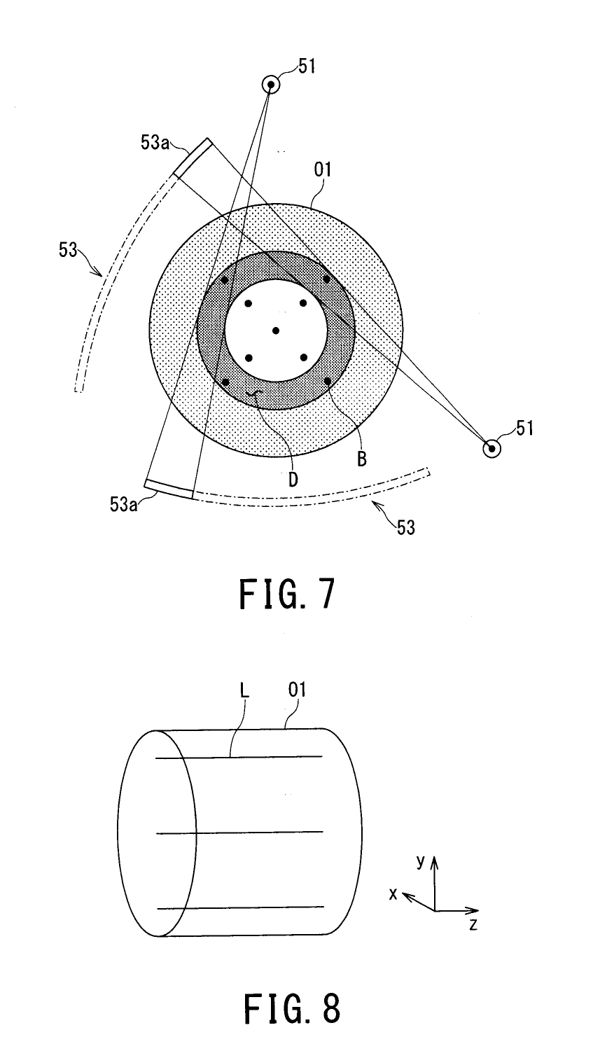

FIG. 7 is an explanatory view illustrating generation of CT images for each detector module.

FIG. 7 illustrates an outermost detector module 53a constituting the X-ray detector 53. When a back projection parameter value in the detector module 53a is calculated, the back projection parameter value is calculated based on the bead CT value on the bead B included in the pre-reconstruction data detected by the detector module 53a. In FIG. 7, the back projection parameter value relating to the detector module 53a is calculated based on the bead CT value of the bead B in a toroidal region D.

Among the CT images on the bead phantom O1, this CT image (toroidal CT image) is generated based only on the pre-reconstruction data acquired by the detector module 53a, and the back projection parameter value is calculated. CT images by other detector modules are similarly generated, and back projection parameter values are calculated in the respective detector modules. In this case, the bead phantom O1 to be used needs to be structured so that at least one bead B is included in the pre-reconstruction data from each detector module.

### (Third Modification)

The phantom scan execution unit 81 illustrated in FIG. 2 scans a line phantom O1 illustrated in FIG. 8. The line phantom O1 contains a plurality of lines L as a plurality of reference substances extending in the slice direction illustrated in FIG. 8.

The lines L and their surrounding substance in the line phantom O1 are so set that the lines L are larger (or smaller) in the absorption coefficient than the surrounding substance. Hereinafter, a description is given of the case where the lines L are larger in the absorption coefficient than the surrounding substance as an example.

The phantom image generation unit 82 illustrated in FIG. 2 generates a plurality of CT images on the line phantom O1 for each of a plurality of slices. The optimum value calculation unit 83 calculates CT values based on the CT images corresponding to the respective back projection parameter values in each slice. The optimum value calculation unit 83 then calculates, for each slice, a back projection parameter value corresponding to a substantially maximum CT value based on the CT values corresponding to each of the back projection parameter values, and defines the generated value as a back projection parameter optimum value. More specifically, the flow chart illustrated in FIG. 5 is executed for each slice.

### (Fourth Modification)

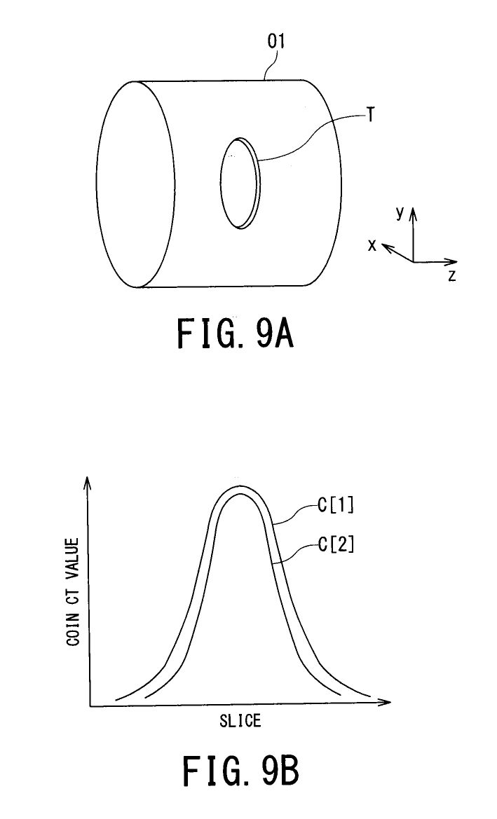

A fourth modification of the X-ray CT apparatus 1 according to the present embodiment aims at enhancing the resolution of CT images in the slice direction. Here, the phantom scan execution unit 81 illustrated in FIG. 2 scans a coin phantom O1 illustrated in FIG. 9A. The coin phantom O1 contains a coin T illustrated in FIG. 9A as a reference substance.

The coin T and its surrounding substance in the coin phantom O1 are so set that the coin T is larger (or smaller) in the absorption coefficient than the surrounding substance.

The phantom image generation unit 82 illustrated in FIG. 2 generates a plurality of CT images on the coin phantom O1 for each of a plurality of slices. The optimum value calculation unit 83 calculates a coin CT value based on the CT image corresponding to each back projection parameter value in each slice. The optimum value calculation unit 83 then generates a graph wherein coin CT values of the respective slices are arranged for each of the back projection parameter values, and calculates a back projection parameter optimum value from the graph.

FIG. 9A is one example of the coin phantom O1, and FIG. 9B is a graph view illustrating the coin CT value in each of the slices.

FIG. 9B illustrates a coin CT value C[1] (equivalent to the value generated in step ST5 illustrated in FIG. 5) corresponding to an FDD value A[1] as a back projection parameter value in each slice of the coin phantom O1 illustrated in FIG. 9A. FIG. 9B illustrates a coin CT value C[2] corresponding to an FDD value A[2] as a back projection parameter value in each slice of the coin phantom O1 illustrated in FIG. 9A. In each of the slices, the coin CT value C[1] is larger than the coin CT value C[2]. More specifically, from the viewpoint of the resolution of an xy plane, the FDD value A[1] corresponding to the coin CT value C[1] is suitable as an optimum value.

However, FIG. 9B indicates that the FDD value A[2] corresponding to the coin CT value C[2] having a smaller half-value width of a slice variation in the coin CT value is suitable as an optimum value from the viewpoint of the resolution in the slice direction.

In the reconstruction processing based on the data on the patient O2, the fourth modification of the X-ray CT apparatus 1 according to the present embodiment generates the CT image on the patient O2 by using the back projection parameter optimum value and the data on the coin phantom O1. Therefore, the fourth modification of the X-ray CT apparatus 1 can enhance the slice-directional resolution of the CT image on the patient O2.

In the reconstruction processing based on the data on the patient O2, the fourth modification of the X-ray CT apparatus 1 generates the CT image by using the back projection parameter optimum value based on the CT value of an image portion (coin portion) in the CT image on the coin phantom O1. Therefore, according to the fourth modification of the X-ray CT apparatus 1, the back projection parameter optimum value can be calculated by using only the CT value of the reference substance (coin). Accordingly, as compared with the case of calculating the back projection parameter optimum value by using the CT value of the entire CT image, the slice-directional resolution of the CT image on the patient O2 can be enhanced with high precision.

### (Fifth Modification)

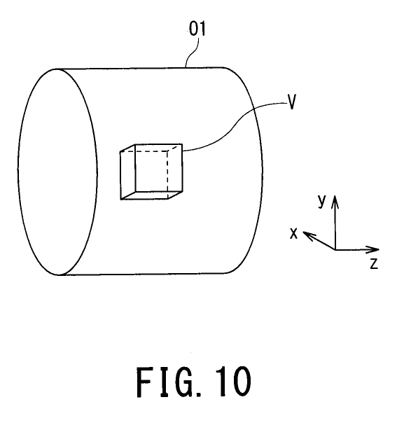

A fifth modification of the X-ray CT apparatus 1 according to the present embodiment aims at enhancing the resolution of CT images in the slice direction as in the fourth modification. Here, the phantom scan execution unit 81 illustrated in FIG. 2 scans a cube phantom O1 illustrated in FIG. 10. The cube phantom O1 contains a cube V in a cubic shape illustrated in FIG. 10 as a reference substance.

The cube V and its surrounding substance in the cube phantom O1 are so set that the cube V is larger (or smaller) in an absorption coefficient than the surrounding substance.

The phantom image generation unit 82 illustrated in FIG. 2 generates a plurality of CT images on the cube phantom O1 for each of a plurality of slices. The optimum value calculation unit 83 calculates a cube CT value based on the CT image corresponding to each of the respective back projection parameter values in each slice. The optimum value calculation unit 83 then generates a graph (equivalent to FIG. 9B) wherein cube CT values of the respective slices are arranged for each of the back projection parameter values, and calculates a back projection parameter optimum value from the graph.

As illustrated in FIG. 9B, from the viewpoint of the resolution of the xy plane, the FDD value A[1] corresponding to the cube CT value C[1] is suitable as an optimum value. However, the FDD value A[2] corresponding to the cube CT value C[2] having a smaller half-value width of a slice variation in the cube CT values is suitable as an optimum value from the viewpoint of the resolution in the slice direction. In that case, one of the optimum values can properly be selected depending on purposes.

In the reconstruction processing based on the data on the patient O2, the fifth modification of the X-ray CT apparatus 1 according to the present embodiment generates the CT image on the patient O2 by using the back projection parameter optimum value and the data on the cube phantom O1. Therefore, the fifth modification of the X-ray CT apparatus 1 can enhance the xy plane resolution and the slice-directional resolution of the CT image on the patient O2.

In the reconstruction processing based on the data on the patient O2, the fifth modification of the X-ray CT apparatus 1 generates the CT image by using the back projection parameter optimum value based on the CT value of an image portion (cube portion) in the CT image on the cube phantom O1. Therefore, according to the fifth modification of the X-ray CT apparatus 1, the back projection parameter optimum value can be calculated by using only the CT value of the reference substance (cube). Accordingly, as compared with the case of calculating the back projection parameter optimum value by using the CT value of the entire CT image, the xy plane resolution and the slice-directional resolution of the CT image on the patient O2 can be enhanced with high precision.

While certain embodiments have been described, these embodiments have been presented by way of example only, and are not intended to limit the scope of the inventions. Indeed, the novel methods and systems described herein may be embodied in a variety of other forms; furthermore, various omissions, substitutions and changes in the form of the methods and systems described herein may be made without departing from the spirit of the inventions. The accompanying claims and their equivalents are intended to cover such forms or modifications as would fall within the scope and spirit of the inventions.

What is claimed is:

1. An X-ray CT apparatus, comprising:
an X-ray tube configured to emit an X-ray;
an X-ray detector configured to detect the X-ray;
a rotation unit configured to rotate at least the X-ray tube around a rotation center;
a data generation unit configured to generate data used in reconstruction processing of a CT image on a basis of an output of the X-ray detector;
an image generation unit configured to perform reconstruction processing of the CT image, the reconstruction processing using the data and a parameter value relating to at least one of a distance (FDD) between a focal position of the X-ray tube and a position of the X-ray detector, and a distance (FCD) between the focal position of the X-ray tube and a position of the rotation center; and
a setting unit configured to set the parameter value used in the reconstruction processing using the data generated when the X-ray is emitted to the object, the parameter value being set based on CT values of a plurality of CT images obtained by a plurality of parameter values used in the reconstruction processing using the data generated when the X-ray is emitted to a phantom.

2. The X-ray CT apparatus according to claim 1, wherein
the setting unit sets, when the phantom contains a reference substance and a surrounding substance surrounding the reference substance, which are different in an absorption coefficient or density, the parameter value based on the plurality of CT images on the phantom so as to cause the CT value corresponding to the reference substance to be a peak.

3. The X-ray CT apparatus according to claim 2, wherein
the setting unit sets, when the phantom contains the surrounding substance and the reference substance larger in the absorption coefficient or the density than the surrounding substance, the parameter value based on the plurality of CT images on the phantom so as to cause the CT value corresponding to the reference substance to be maximum.

4. The X-ray CT apparatus according to claim 3, wherein
the setting unit sets, when the phantom contains the plurality of reference substances and the surrounding substance, the parameter value based on the plurality of CT images on the phantom so as to cause a total value or an average value of the CT values corresponding to the plurality of reference substances to be maximum.

5. The X-ray CT apparatus according to claim 2, wherein
the setting unit sets, when the phantom contains the surrounding substance and the reference substance smaller in the absorption coefficient or the density than the surrounding substance, the parameter value based on the plurality of CT images on the phantom so as to cause the CT value corresponding to the reference substance to be minimum.

6. The X-ray CT apparatus according to claim 5, wherein
the setting unit sets, when the phantom contains the plurality of reference substances and the surrounding substance, the parameter value based on the plurality of CT images on the phantom so as to cause a total value or an average value of the CT values corresponding to the plurality of reference substances to be minimum.

7. The X-ray CT apparatus according to claim 1, wherein
the parameter value further includes at least one of a focal point size of the X-ray tube, a channel direction position of each detection element included in the X-ray detector, an X-ray incidence direction position of the each detection element, a column direction position of the each detection element, and an in-plane rotation direction position of the each detection element.

8. The X-ray CT apparatus according to claim 1, wherein
the image generation unit performs the reconstruction processing using the data and the parameter value corresponding to an angle of rotation by the rotation unit.

9. The X-ray CT apparatus according to claim 1, wherein
the image generation unit generates, when an operation to change the parameter value is performed after the CT image is displayed, a CT image by using a parameter value after changed.

10. An X-ray CT apparatus, comprising:
an X-ray tube configured to emit an X-ray;
an X-ray detector configured to detect the X-ray emitted by the X-ray tube;
a data generation unit configured to generate data used in reconstruction processing of a CT image on a basis of an output of the X-ray detector;
an image generation unit configured to perform reconstruction processing of the CT image, the reconstruction processing using the data and a parameter value; and
a setting unit configured to set the parameter value used in the reconstruction processing using the data generated when the X-ray is emitted to the object, the parameter value being set based on CT values of a plurality of CT images obtained by a plurality of parameter values used in the reconstruction processing using the data generated when the X-ray is emitted to a phantom.

11. A method for image processing, comprising:
obtaining data used in reconstruction processing of a CT image from a storage, the data being acquired by a CT scan;
performing reconstruction processing of the CT image, the reconstruction processing using the data and a parameter value relating to at least one of a distance (FDD) between a focal position of an X-ray tube configured to emit an X-ray and a position of an X-ray detector configured to detect the X-ray, and a distance (FCD) between the focal position of the X-ray tube and a position of the rotation center; and
setting the parameter value used in the reconstruction processing using the data generated when the X-ray is emitted to the object, the parameter value being set based on CT values of a plurality of CT images obtained by a plurality of parameter values used in the reconstruction processing using the data generated when the X-ray is emitted to a phantom.

12. The method for image processing according to claim 11, wherein
the setting sets, when the phantom contains a reference substance and a surrounding substance surrounding the reference substance, which are different in an absorption coefficient or density, the parameter value based on the plurality of CT images on the phantom so as to cause the CT value corresponding to the reference substance to be a peak.

13. The method for image processing according to claim 12, wherein
the setting sets, when the phantom contains the surrounding substance and the reference substance larger in the absorption coefficient or the density than the surrounding substance, the parameter value based on the plurality of CT images on the phantom so as to cause the CT value corresponding to the reference substance to be maximum.

14. The method for image processing according to claim 13, wherein
the setting sets, when the phantom contains the plurality of reference substances and the surrounding substance, the parameter value based on the plurality of CT images on the phantom so as to cause a total value or an average value of the CT values corresponding to the plurality of reference substances to be maximum.

15. The method for image processing according to claim 12, wherein
the setting sets, when the phantom contains the surrounding substance and the reference substance smaller in the absorption coefficient or the density than the surrounding substance, the parameter value based on the plurality of CT images on the phantom so as to cause the CT value corresponding to the reference substance to be minimum.

16. The method for image processing according to claim 15, wherein
the setting sets, when the phantom contains the plurality of reference substances and the surrounding substance, the parameter value based on the plurality of CT images on the phantom so as to cause a total value or an average value of the CT values corresponding to the plurality of reference substances to be minimum.

17. The method for image processing according to claim 11, wherein
the parameter value further includes at least one of a focal point size of the X-ray tube, a channel direction position of each detection element included in the X-ray detector, an X-ray incidence direction position of the each detection element, a column direction position of the each detection element, and an in-plane rotation direction position of the each detection element.

18. The method for image processing according to claim 11, wherein
the image generation performs the reconstruction processing using the data and the parameter value corresponding to an angle of rotation by the rotation unit.

19. The method for image processing according to claim 11, wherein
the image generation generates, when an operation to change the parameter value is performed after the CT image is displayed, a CT image by using a parameter value after changed.
